(19) United States
(12) Patent Application Publication
(10) Pub. No.: US 2024/0335319 A1
(43) Pub. Date: Oct. 10, 2024

(54) METHOD AND APPARATUS FOR ON DEMAND TEAR PRODUCTION

(71) Applicant: Karen Stein, Woodland Hills, CA (US)

(72) Inventor: Karen Stein, Woodland Hills, CA (US)

(21) Appl. No.: 18/658,679

(22) Filed: May 8, 2024

Related U.S. Application Data
(60) Provisional application No. 63/457,060, filed on Apr. 4, 2023.

(51) Int. Cl. A61F 9/00

(52) U.S. Cl. CPC A61F 9/0008

(57) ABSTRACT

The disclosed method and apparatus are unique to an individual end-user and address concerns with cross-contamination and germ transmission when triggering on demand tear production. The disclosed device is customizable to each end-user, is portable and easily stored. Each component of the device can be modified to fit the needs and requirements of each end user. In addition, the fume generating substance is not limited to one substance but is likewise customizable to produce the desired result of on-demand tear production.

### CROSS-REFERENCE TO RELATED PATENT APPLICATIONS

The present application is a non-provisional application claiming priority to U.S. Provisional Patent Application Ser. No. 63/457,060, filed Apr. 4, 2023.

### BACKGROUND OF THE INVENTION

### 1. Field of the Invention

Embodiments of the invention relate to a method and apparatus for improving on demand tear production, and more specifically the method and apparatus provides a device for self-application of fumes initiating tear production.

### 2. Description of Related Art

Film and television makeup artists often need to assist an on-screen actor with generating tears for crying scenes. Traditionally, this was done with a device that allows the makeup artist to blow menthol fumes into the actor's eye irritating the tear duct and causing tears. Though this method has always been questionable in context of sanitation (blowing breath into another person's eye), since returning to production after the COVID-19 shutdown, it is completely unacceptable to those in the industry. The inventive concept addresses this concern and allows the actor to blow the fumes into his/her eye, thereby circumventing any issues with sanitation and germ transmission.

### SUMMARY OF THE INVENTION

The disclosed method and apparatus is unique to an individual end-user and therefore addresses concerns with cross-contamination and germ transmission. The device is customizable to each end-user, is portable and easily stored. Each component of the device can be modified to fit the needs and requirements of each end user. In addition, the fume generating substance is not limited to one substance but is likewise customizable to produce the desired end result of on demand tear production.

### BRIEF DESCRIPTION OF THE DRAWINGS

FIG. 1 shows a disassembled exemplary embodiment of the device; and

FIG. 2 shows an assembled exemplary embodiment of the device.

### DETAILED DESCRIPTION OF EMBODIMENTS

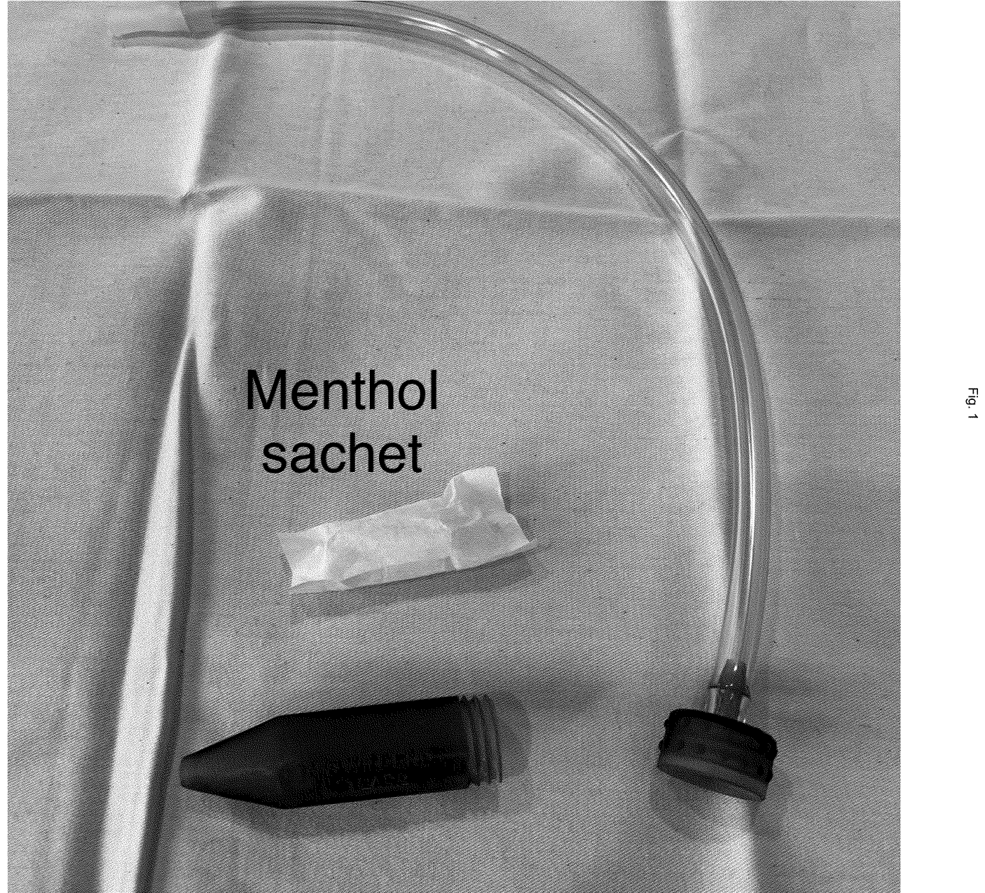

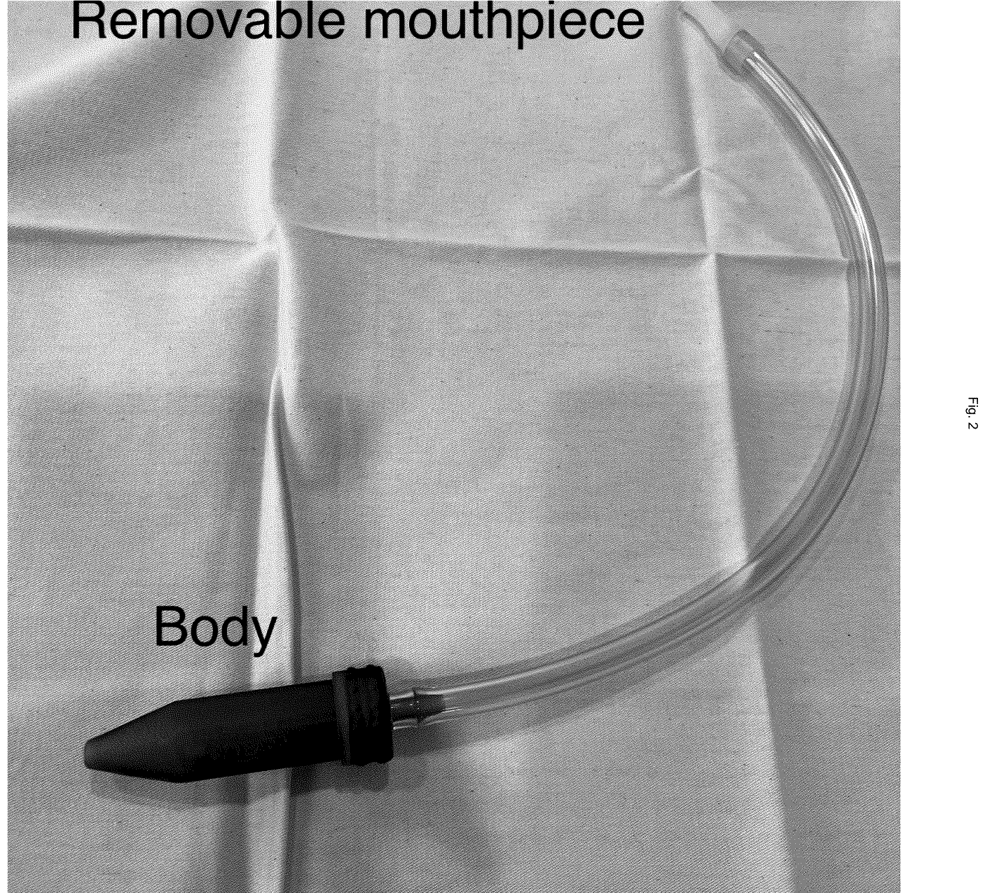

While the invention is illustrated and described in a preferred embodiment, the invention may be produced and described in many different configurations, forms and various methods, without deviating from the scope of the present invention. The preferred embodiment of the invention is described in detail below with reference to the accompanying FIGS. 1-2, with the understanding that the present disclosure is to be considered as an exemplification of the principles of the invention and the associated functional specifications for its construction and is not intended to limit the invention to the embodiment illustrated. Those skilled in the art will envision many other possible variations within the scope of the invention described herein.

The apparent diameters of the sections in FIG. 1-FIG. 2 are greatly exaggerated relative to their apparent lengths to clarify the relationships of the components. The illustrations are provided for the purpose of describing a preferred embodiment of the invention and are not intended to limit the invention thereto.

Referring now to FIGS. 1 and 2, a mouthpiece is shown. The mouthpiece may be a silicone tip that is comfortable to use, removable for cleaning or disposal, and comes in a variety of colors and/or sizes for customization to a particular end user. Alternative embodiments may include a mechanical airflow-generating device, in which case the mouthpiece may be fixed, or remain removable, as breath contamination is no longer a concern. The mechanical airflow-generating device may be a small motor powering a fan or similar device and is well known to those of ordinary skill in the art. The mechanical airflow-generating device may also be a bulb composed of silicone or other flexible material.

FIGS. 1 and 2 also depict a tube, which may be a clear PVC type tube that connects the mouthpiece and the body of the blower. The tube is easily removable and replaceable as needed. The tube can be of varying lengths to accommodate the end user requirements and may be adjustable. In other embodiments, such as when the mechanical airflow-generating device is a small motor powering a fan or a bulb composed of a flexible material, the tube may be of a small length or not present.

FIGS. 1 and 2 also depict a body of the device, which is a hollow vessel to house the fume generating substance. The body can be of varying lengths dependent upon the type of substance from which fumes are generated. In an embodiment, the body is about 2.5 inches long. In addition, the body may have a screw-on cap to facilitate inserting the fume generating substance to the hollow portion of the body and includes a pointed end to concentrate the fumes before application to the end user's eyes. In alternative embodiments, the body may include a screening device to filter foreign objects and/or substances before the fumes are blown into the end user's eye.

FIGS. 1 and 2 also depict a non-limiting example of a fume generating substance. In an embodiment, the fume generating substance is a menthol sachet as depicted in FIGS. 1 and 2. The menthol sachet includes menthol crystals secured in a permeable sachet sealed on all sides to prevent any pieces of menthol crystal from being blown into the end user's eyes. In alternative embodiments, the fume generating substance may be raw menthol crystals secured in the body with cotton, camphor salve deposited on a cotton ball, menthol or camphor oil on a cotton ball, or vapor cartridges known to those of ordinary skill in the art. The fume generating substance can be any substance known in the art capable of safely producing fumes used to generate tears from an eye of the end user.

The invention is small and portable and can easily be stored and carried by a makeup artist. The device can therefore be ready on demand to assist in tear generation within a few seconds. The disclosed device addresses the concern of cross-contamination or germ transmission from third-parties as it is unique to the particular end user.

The principles, embodiments, and modes of operation of the present invention have been set forth in the foregoing specification. The embodiments disclosed herein should be interpreted as illustrating the present invention and not as restricting it. The foregoing disclosure is not intended to limit the range of equivalent structure available to a person of ordinary skill in the art in any way, but rather to expand the range of equivalent structures in ways not previously contemplated. Numerous variations and changes can be made to the foregoing illustrative embodiments without departing from the scope and spirit of the present invention.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent particular aspects and embodiments of the invention and are therefore representative examples of the subject matter that is broadly contemplated by the present invention. It is further understood that the scope of the present invention fully encompasses other embodiments that are, or may become, obvious to those skilled in the art and that the scope of the present invention is accordingly not limited by the descriptions presented herein.

I claim:

1. A method for on demand tear production, the steps consisting of:
selection of a preferred fume generating substance;
customization of the device as depicted in FIGS. 1 and 2 to conform to the particular needs of an end user;
generating fumes from the fume generating substance selected;
concentrating the generated fumes to an eye of the end user; and
creation of tears on demand.

2. An apparatus comprising:
an air intake component;
a chamber containing a substance that will produce vapors that will induce tearing in a human eye that is connected at a first end to the air intake component in an airtight manner; and
an air outflow component connected to a second end of the chamber in an airtight manner.

3. The apparatus of claim 2, wherein the air intake component is comprised of a mouthpiece open at both ends that is connected to the chamber at one end.

4. The apparatus of claim 3, wherein the mouthpiece is constructed of an inflexible tube.

5. The apparatus of claim 2, wherein the air intake component comprises:
a. A mouthpiece open at both ends;
b. Tubing that is connected at a first end to the mouthpiece in an airtight manner and connected to the chamber at a second end in an airtight manner.

6. The apparatus of claim 5, wherein the tubing is constructed of a flexible material.

7. The apparatus of claim 5, wherein the tubing is constructed of an inflexible material.

8. The apparatus of claim 2, wherein the air intake component is a container open at one end and constructed of a flexible material.

9. The apparatus of claim 2, wherein the substance is selected from a group consisting of menthol, camphor, or a mixture of menthol and camphor.

10. The apparatus of claim 9, wherein the substance is held in a packet permeable to atmosphere and the packet is held in the container.

11. The apparatus of claim 2, wherein the chamber is constructed of inflexible tubing.

12. The apparatus of claim 11, wherein the chamber is constructed of plastic.

13. The apparatus of claim 11, wherein the chamber is constructed of metal.

14. The apparatus of claim 2, wherein the chamber comprises:
a screw cap with one end attached to the air intake component via an airtight method and a second end with internal threading;
a container with one end removable attached with external threading to the screw cap and a second end.

15. The apparatus of claim 2, wherein the air outflow component is a graduated tube, open at both ends, with the larger end connected to the second end of the chamber in an airtight manner, and the smaller end open to the air.

16. The apparatus of claim 2, wherein the air outflow component is removable and contains a filtration material.

17. The apparatus of claim 2, wherein the air outflow component contains a filtration material.
